(19) United States
(12) Patent Application Publication
(10) Pub. No.: US 2010/0305436 A1
(43) Pub. Date: Dec. 2, 2010

(54) SYSTEMS, DEVICES, AND METHODS FOR PHOTOACTIVE ASSISTED RESECTION

(75) Inventors: James C. Chen, Clyde Hill, WA (US); Llew Keltner, Portland, OR (US)

Correspondence Address: SEED INTELLECTUAL PROPERTY LAW GROUP PLLC, 701 FIFTH AVE, SUITE 5400, SEATTLE, WA, 98104, US

(73) Assignee: Light Sciences Oncology , Inc., Bellevue, WA (US)

(21) Appl. No.: 12/678,123

(22) PCT Filed: Sep. 12, 2008
(86) PCT No.: PCT/US2008/076320

§ 371 Date: Aug. 9, 2010

Related U.S. Application Data
(60) Provisional application No. 60/993,915, filed on Sep. 14, 2007.

(51) Int. Cl. A61B 6/00

(52) U.S. Cl. 600/431

(57) ABSTRACT

A method and system is used to locate tissue for removal during a resection. The system includes a container that holds a photoactivatable agent capable of binding with unwanted tissue at a treatment site, a light source, and a viewing device. The light source is adapted to continuously output light that causes fluorescence of the photoactivatable agent in unwanted tissue. The system provides fluorescence-enhanced viewing of the treatment site through the viewing device. The viewing device has a filter that filters out light from the light source that is reflected from the treatment site while allowing a fluorescence emission from the photoactivatable agent to pass therethrough to each eye of the user. The light from the light source and the fluorescence emission are in different regions of the energy spectrum.

### CROSS-REFERENCE TO RELATED APPLICATION

This application claims the benefit under 35 U.S.C. §119(e) of U.S. Provisional Patent Application No. 60/993,915, filed Sep. 14, 2007, which is incorporated herein by reference in its entirety.

### BACKGROUND

1. Technical Field

The present invention relates generally to systems, devices, and methods for performing medical procedures, such as resections, using a photoactivatable agent.

2. Description of the Related Art

Medical procedures are often performed to remove unwanted tissue. Resections are types of medical procedures that involve surgery to remove tissue, such as tumors. Tumor margins can be difficult to locate during a resection, thus making it difficult to remove malignant tissue while sparing normal tissue (e.g., nonmalignant tissue), organs, or other anatomical features. Sparing normal tissue is often advantageous to maintain important bodily functions, especially when an en bloc resection is not feasible. For example, when performing brain surgery, a surgeon tries to remove all of the cancerous tissue while leaving the healthy, non-cancerous tissue. Complete removal of tumors may significantly increase survival rates and survival times of the subjects. If an entire tumor is not removed, the portion of the tumor left in the subject may continue to spread. Spared healthy tissue can continue to function to maximize brain function, thereby reducing adverse effects associated with the surgical procedure. Standard white light used to illuminate the brain tissue does not enhance the margins of the tumors. It may be difficult or impossible to visually determine borders of tumors, even when using a surgical microscope (e.g., a neurosurgical microscope), loupes, or other optical aids.

### BRIEF SUMMARY

A photoactive assisted resection system includes a photoactivatable agent and a light source for activating the agent. The system is used to aid in the identification of tissue. The system, in some embodiments, provides or enhances visual differentiation between different types of tissue, such as diseased tissue, malignant tissue, healthy tissue, or the like. During a resection, a user can observe the surgical site using the system in order to determine where to remove unwanted tissue while sparing as much healthy tissue as possible.

A photoactive assisted resection system, in some embodiments, includes a viewing device, a fluorescing agent (e.g., a photoactivatable agent, photosensitizer, etc.), and a light source capable of exciting the fluorescing agent. The viewing device includes an optical filter adapted to at least partially attenuate an excitation wavelength(s) or waveband(s) such that the user visually identifies fluorescence of the agent. The fluorescence provides enhanced visual contrast between targeted tissue to be removed and non-targeted tissue to be left in the subject. In some embodiments, the light source is configured to provide generally constant excitation of the agent for continuous fluorescence.

The light source includes, without limitation, one or more light-emitting diodes (LEDs), lasers, halogen light sources, incandescent sources, or other non-coherent or coherent light sources. In some embodiments, the light source includes a light emitter, such as a lamp, LED, laser, or other light generator, and one or more filters. For example, the light source can be a light emitter (e.g., a halogen lamp or the like) that outputs white light and a filter that transmits certain wavelength(s) or waveband(s). The light source's excitation wavelength(s), peak emission, or waveband(s) can be matched to absorption wavelength(s) or waveband(s) of the agent. The light intensity, pulse sequence (if any), direction, size and shape of illumination field from the light source may be determined, either empirically or modeled mathematically, to yield the desired information. If the light source is a broadband light source, the optical filter may be used to attenuate, select, and/or modify substantially all or a part of the light source's emission spectrum to allow for easier detection of the fluorescence associated with the agent, or other measurable or visible characteristics. In some embodiments, the light source is part of a head-lamp assembly suitable for use with goggles or other viewing devices, such as glasses, loupes, or the like. In some embodiments, the light source is an array or panel of light sources (e.g., blue and/or red LEDs).

The light source can be a separate device or can be incorporated into a resection instrument or tool. The light source can be mated, connected, or integral to the resection instrument or tool. The light source can optionally be mated with or coupled to a controller, signal detector, magnification system, and/or other type of viewing device. Fluorescence can be detected visually or through use of a detector, such as a spectrometer type detector. Such a detector may have audible or visual means of conveying signal strength information to a user. The system may further be incorporated into an instrument (e.g., an endoscope, a bronchoscope, and the like) and used during, for example, an open, semi-open, or closed operation.

The viewing device, in some embodiments, is used to view, evaluate, diagnose, or otherwise analyze the subject's tissue. In some embodiments, the viewing device is eyewear (e.g., goggles or glasses) configured to transmit the fluorescence of the agent and attenuate (e.g., partially or completely block) other light, such as at least some ambient light or reflected light, or both. In some embodiments, the viewing device is a filter attachment for coupling to a device (e.g., a microscope, a telescope, or a loupe) used in surgery.

The fluorescing agent is administered topically, intravenously, transdermally, intra-arterially, orally, or interstitially via an injection (e.g., injecting directly into the tissue). The fluorescing agent can also be a photoactivatable agent delivered using liposomal techniques, transdermal techniques, and/or iontophoretic techniques. After, during, or before administration of the selected agent in an appropriate dose, the light source is positioned outside of the patient at a preselected location appropriate to illuminate a treatment site.

The fluorescing agent can be administered prior to illumination. After a period allowing for agent uptake by a tumor or other targeted tissue, the light source is activated to illuminate the treatment site. A fluorescence signal is detected by direct visualization or utilizing an aid, such as a microscope and/or a detector and amplifier. If the signal is above a certain threshold, the tissue is removed mechanically, vaporized, or ablated. As the next layer or location is visualized and excited by the light source, the process is repeated. In some embodiments, the process is repeated until no further fluorescence is detected, or the signal falls below a defined threshold. In this manner, the system can be used to accurately and precisely treat (e.g., remove, vaporize, ablate, and the like) undesired tissue.

In some embodiments, a system for viewing targeted tissue at a treatment site comprises a photoactivatable agent, a light source, and a direct-viewing device. The direct-viewing device for observing the treatment site therethrough includes a filter adapted to filter out light while allowing a portion or most of the fluorescence emission from the photoactivatable agent to pass therethrough to one or both eyes of a user to increase contrast between the targeted tissue and non-targeted tissue as the light source outputs light.

In some embodiments, a system for viewing targeted tissue at a treatment site comprises a photoactivatable agent adapted to preferentially associate with targeted tissue, a light source, and a direct-viewing device. The light source is adapted to output light that causes the photoactivatable agent to produce a fluorescence emission when the photoactivatable agent is associated with the targeted tissue at the treatment site. The direct-viewing device for observing the treatment site therethrough includes a filter adapted to filter out light from the light source that is reflected from the treatment site while allowing the fluorescence emission from the photoactivatable agent to pass therethrough to one or both eyes of a user to increase contrast between the targeted tissue and non-targeted tissue as the light source outputs light.

The photoactivatable agent, in some embodiments, is adapted to cause greater fluorescence of targeted tissue in which the photoactivatable agent has accumulated than the non-targeted tissue when both the targeted tissue and the non-targeted tissue are illuminated by the light source. In some embodiments, the light source is adapted to emit non-coherent light. In other embodiments, the light source is adapted to emit coherent light. In some embodiments, the light source comprises at least one light emitter and filter, a light-emitting diode, and/or lamp capable of outputting mostly blue light that causes the photoactivatable agent to fluoresce red light. In some embodiments, the direct-viewing device has imaging optics configured to provide three-dimensional viewing of the treatment site. In some embodiments, the imaging optics magnify an image of the treatment site viewed through the direct-viewing device. In some embodiments, the direct-viewing device includes eyewear wearable by the user. In some embodiments, the direct-viewing device includes a pair of loupes, a surgical telescope, and/or a surgical microscope. In some embodiments, the filter comprises a bandpass filter. In some embodiments, the filter is a clip-on filter adapted to couple to a microscope and/or eyewear. In some embodiments, the light source is adapted to continuously output light that causes the photoactivatable agent to produce a continuous fluorescence emission. The light source can be coupled to a controller.

A resection kit for performing surgery comprises instructions for use and packaging. The packaging is capable of holding the instructions for use and a system for viewing targeted tissue at a treatment site. In some embodiments, the kit further includes a surgical tool configured to remove unwanted tissue identified by viewing through a direct-viewing device of the system.

In some embodiments, a system for viewing tissue at a surgical site includes a photoactivatable agent adapted to fluoresce and an external viewing device through which a user is capable of observing the surgical site. The viewing device includes a passive filter that transmits light associated with fluorescence of the photoactivatable agent and that filters out other light to increase the relative amount of the light associated with the fluorescence delivered to each eye of the user for true fluorescence-enhanced viewing of the surgical site enabling optical differentiation of targeted tissue for removal and other tissue at the surgical site.

The viewing device, in some embodiments, further includes imaging optics configured to magnify an image of the surgical site and to allow rays of light from the surgical site to pass therethrough to the user's eyes. In some embodiments, the external viewing device is adapted to provide an analog image of the surgical site. In some embodiments, the external viewing device is wearable goggles or a clip for coupling to a microscope. In some embodiments, a light source is coupled to the imaging optics. In some embodiments, the system further comprises a resection tool carrying a light source adapted to cause fluorescence of the photoactivatable agent. In some embodiments, the system further comprises a resection tool carrying a filter.

In some embodiments, a system for viewing tissue at a surgical site comprises a photoactivatable agent adapted to fluoresce and a resection tool. The resection tool includes a passive filter that transmits light associated with fluorescence of the photoactivatable agent and that filters out other light to increase the relative amount of light associated with the fluorescence delivered to the user for true fluorescence-enhanced viewing of the surgical site enabling optical differentiation of targeted tissue for removal and other tissue at the surgical site.

In other embodiments, a method of viewing targeted tissue at a treatment site is provided. The method includes delivering a photoactivatable agent to the treatment site of a subject. The photoactivatable agent at the treatment site is exposed to light from an energizable light source to cause fluorescence of the photoactivatable agent at the treatment site. A fluorescence-enhanced image of the treatment site is directly viewed by looking with one or both eyes through a viewing device. The viewing device includes a filter adapted to filter out light from the energizable light source that is reflected from the treatment site while allowing fluorescence to pass therethrough to one or both eyes of the user. The targeted tissue is identified based at least in part on directly viewing of the fluorescence-enhanced image.

In some embodiments, the energizable light source is a light-emitting diode or panel positioned external to the subject. In some embodiments, exposing the photoactivatable agent at the treatment site to light comprises continuously emitting non-coherent light that illuminates the treatment site. In some embodiments, each eye of the user directly views the fluorescence-enhanced image. In some embodiments, margins of cancerous tissue are located by comparing visual differences between the cancerous tissue and non-cancerous tissue. In some embodiments, cancerous tissue at the treatment site is removed based, at least in part, on the identification of cancerous tissue. In some embodiments, the photoactivatable agent accumulates in cancerous tissue such that a concentration of the photoactivatable agent in the cancerous tissue is greater than a concentration of photoactivatable agent in non-cancerous tissue. In some embodiments, the photoactivatable agent is configured to selectively accumulate in cancerous tissue and to not accumulate in non-cancerous tissue. In some embodiments, a sufficient amount of time is allowed to pass for photoactivatable agent that is not accumulated to the cancerous tissue to clear from the treatment site prior to identifying the targeted tissue. In some embodiments, the fluorescence-enhanced image has a greater amount of fluorescence from the photoactivatable agent than an amount of fluorescence from the photoactivatable agent viewed by a naked eye.

In yet other embodiments, an apparatus is adapted to view targeted tissue at a treatment site. The apparatus is adapted to expose a photoactivatable agent at the treatment site to light from an energizable light source to cause fluorescence of the photoactivatable agent. A fluorescence-enhanced image of the treatment site is directly viewed by looking with both eyes through a viewing device. The viewing device, in some embodiments, includes a filter adapted to filter out light from the energizable light source reflected from the treatment site while allowing fluorescence to pass therethrough to each eye. The targeted tissue is identified based at least in part on directly viewing of the fluorescence-enhanced image.

The various components of the systems can be disposable, reusable, sterilized, or combinations thereof. For example, the systems can include one or more disposable components and one or more reusable components. The components may be packaged in sterile packaging to prevent or limit contamination. Sterilized components can be used in a wide range of different surgical procedures.

In some embodiments, an external viewing device through which a user is capable of observing the surgical site includes a filter that both transmits light emitted by the photoactivatable agent and that filters out other light to enable direct-viewing and fluorescence-enhanced optical differentiation of targeted tissue and/or other tissue. In some embodiments, the direct-viewing provides three-dimensional viewing of tissue, including both the targeted and non-targeted tissue. The contours and location of tissue can be conveniently evaluated. The direct viewing can be used to accurately view tissue in real-time. Fluorescence-enhanced optical differentiation can enable visual differentiation between different tissues based on, for example, the presence or concentration of a drug. In some embodiments, incident light from a light source is used to selectively excite the drug such that the drug emits light used to provide fluorescence-enhanced optical differentiation.

### BRIEF DESCRIPTION OF THE SEVERAL VIEWS OF THE DRAWINGS

The following drawings are intended as an aid to an understanding of the invention and to present examples of the invention, but do not limit the scope of the invention as described and claimed herein. In the drawings, identical reference numbers identify similar elements or acts.

FIG. 1 is a pictorial view of a system for identifying targeted tissue at a treatment site, in accordance with one illustrated embodiment.

FIG. 2 is a cross-sectional view of an optical element of the system of FIG. 1, in accordance with one illustrated embodiment.

FIG. 3 is a flow chart of one method for viewing a treatment site using the system of FIG. 1.

FIG. 4 is a pictorial view of goggles for use with a photoactivatable agent, in accordance with one illustrated embodiment.

FIG. 5 is a cross-sectional view of the goggles of FIG. 4 taken along line 5-5.

FIG. 6 is a plan view of a microscope for use with a photoactivatable agent, in accordance with one illustrated embodiment.

FIG. 7 is a surgical kit for use with a microscope, in accordance with one illustrated embodiment.

FIG. 8 is a surgical kit having a lamp, a photoactivatable agent, and a viewing device, in accordance with one embodiment.

### DETAILED DESCRIPTION

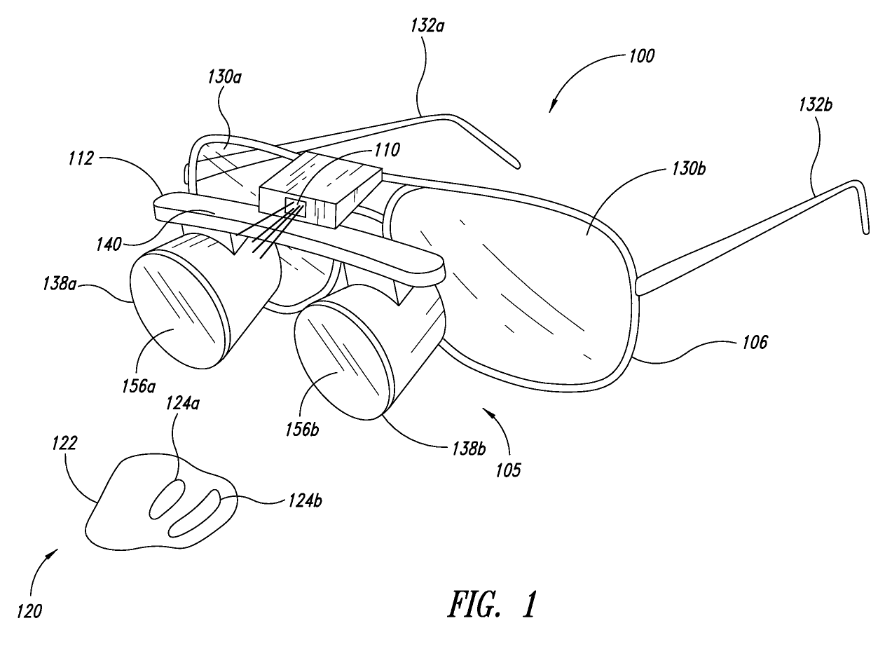

FIG. 1 shows a system 100 for performing photoactive assisted procedures. The system 100 can help the user visually differentiate non-targeted tissue 122 from targeted tissue 124a, 124b (collectively 124) for diagnostic applications or therapeutic applications, or both. The system 100 includes a viewing device 105, illustrated as a direct-viewing device, and a light source 110 carried by the viewing device 105. The illustrated light source 110 illuminates a treatment site 120 in which a photoactivatable agent is dispersed to cause either the targeted tissue 124 or the non-targeted tissue 122 to fluoresce, thereby enabling visual differentiation of these tissues. In this manner, the user can view the treatment site in real-time to obtain information about the subject's tissue ex vivo or in vivo. The illustrated viewing device 105 includes eyewear 106 and an optical aid 112 permanently or temporarily coupled to the eyewear 106.

After the photoactivatable agent is delivered to the treatment site 120, the light source 110 illuminates the entire site 120 to cause the agent to become excited to produce a fluorescence emission. Based on the fluorescence emission, the user can visually obtain information about the site 120. The viewing system 100, in some embodiments, attenuates wavelength(s) and/or waveband(s) to increase the visibility of the fluorescence emission, to increase the contrast between tissues containing different amounts of the photoactivatable agent, to intensify the fluorescence emission, combinations thereof, or the like. Based on the location and size of the targeted tissue 124, the type of tissue surrounding the tissue 124, desired cutting locations, adjacent anatomical features, or other criteria known in the art, a physician can determine an appropriate course of action.

In some diagnostic applications, the light source 110 can output a wavelength or waveband selected to cause the administered photoactivatable agent to fluoresce in order to acquire information about tissue, with or without damaging the tissue. The agent can be self-administered by the subject or can be administered by a nurse, physician, or other suitable individual. Appropriate treatment can then be selected based, at least in part, on the acquired information.

In some therapeutic applications, the output from the light source 110 causes the agent to undergo a reaction, such as a photochemical reaction, to cause localized tissue or cell damage, such as a cell lysis, apoptosis of tissue, or necrosis of tissue. A resection procedure can include both surgically removing tissue and damaging other tissue using such photochemical reactions. Some procedures may involve both diagnostic and therapeutic applications.

With continued reference to FIG. 1, the system 100 can be used to perform a wide range of procedures. If the treatment site 120 is a portion of the brain and the targeted tissue 124 is cancerous brain tissue (e.g., gliomas), the system 100 can be used to accurately locate and remove the targeted tissue 124. Margins of the cancerous tissue 124 can be readily identified in order to remove substantially all of the cancerous tissue 124 while minimizing or limiting the removal of healthy, non-cancerous tissue 122. In some embodiments, a surgeon views a true fluorescence-enhanced image provided by the system 100 in order to identify and remove a desired amount of the unwanted tissue 124 without performing time consuming medical tests, such as a biopsy. The optical aid 112 provides attenuation of light to produce the true fluorescence-enhanced image. The illustrated optical aid 112 also provides magnified viewing for visually identifying relatively small features (e.g., blood vessels, nerve tissue, or other small anatomical features) that are not readily visible to the naked eye.

The eyewear 106 of FIG. 1 is in the form of glasses with lenses 130a, 130b (collectively 130). Arms 132a, 132b of the eyewear 106 can be placed upon the wearer's right and left ears, respectively. The eyewear 106 can be comfortably worn for extended lengths of time while the optical aid 112 is positioned along a line of sight of the wearer. The eyewear 106 can therefore be worn during most or all of a surgical procedure.

The light source 110 can include one or more LEDs (such as edge emitting LEDs, surface emitting LEDs, super luminescent LEDs, or the like), laser diodes, electroluminescent light sources, incandescent light sources, cold cathode fluorescent light sources, organic polymer light sources, lamps, inorganic light sources, or other suitable light emitting sources. U.S. Pat. No. 6,580,228, which is incorporated by reference in its entirety, discloses various types of light sources, such as lamps, that can be utilized. The illustrated light source 110 can emit appropriate wavelength(s) and/or waveband(s) suitable for eliciting a desired response from the photoactivatable agent. The emitted radiation wavelength(s) or waveband(s) can correspond with, or at least overlap with, the wavelength(s) or waveband(s) that excite or otherwise activate the photoactivatable agent so as to fluoresce. In some embodiments, the spectral bandwidth of the light sources provides a significant overlap with the absorption curve of the photoactivatable agent.

The illustrated optical aid 112 includes a pair of passive filters 156a, 156b (collectively 156) through which a right eye and a left eye of the user, respectively, observe the site 120. The filters 156 attenuate light from the light source 110 that is reflected from the site 120 while continuously allowing a fluorescence emission from the administered photoactivatable agent to pass therethrough to the user's eyes. At least one eye of the user views an enhanced contrast image of the site 120. The visible contrast between the non-targeted tissue 122 and the targeted tissue 124 is greater than the contrast viewed by the naked eye or with conventional neurosurgical microscopes, goggles, or loupes. In some embodiments, the optical aid 112 provides the true fluorescence-enhanced image in which the relative amount of fluorescence to other light (e.g., reflected light and/or ambient light) is increased. The user can observe bright fluorescent regions corresponding to the targeted tissue 124 and the surrounding non-fluorescing non-targeted tissue. The borders of the bright fluorescing tissue 124 are clearly visible and distinguishable from non-targeted tissue, including adjacent tissue and remote tissue which does not contain a significant amount of the agent.

The signal-to-noise ratio can be adjusted to ensure that the observer can identify the tissue margins in real-time. Because the image is a true image and not processed (i.e., not a digitized image), the user views a sharp, undistorted image of the entire site 120. Both eyes of the user can view similar images to minimize, limit, or substantially prevent problems associated with viewing dissimilar images. For example, these similar images can have approximately the same amount of visible fluorescence. If the site 120 is viewed by the naked eye (e.g., without the optical device 112), the borders of the tissue 124 may not be visible or may be difficult to locate because of the reflected light and/or at least some ambient light. The optical aid 112 can thus provide viewing with significantly increased visible fluorescence.

The illustrated optical aid 112 of FIG. 1 includes a pair of loupes 138a, 138b (collectively 138) that can be generally similar to each another and, accordingly, the description of one of the loupes applies equally to the other, unless clearly indicated otherwise. In various embodiments, the loupes 138 include imaging optics to magnify an image. Light propagates through the imaging optics so as to produce observable magnified images. Imaging optics include, without limitation, one or more prisms, beam splitters, mirrors, and/or lenses, such as objective lenses, compound lenses, eyepiece lenses, or convex lenses (e.g., biconvex lenses, piano-convex lenses, or the like). Components of the imaging optics cooperate to provide different types of images, such as real images or virtual images. In some embodiments, the viewable image is formed mostly or entirely of light rays that travel through the loupes 138, thereby providing a true image without distortions and inaccuracies often associated with digitized images. The loupes 138 can employ stationary optical elements that are not prone to malfunctions.

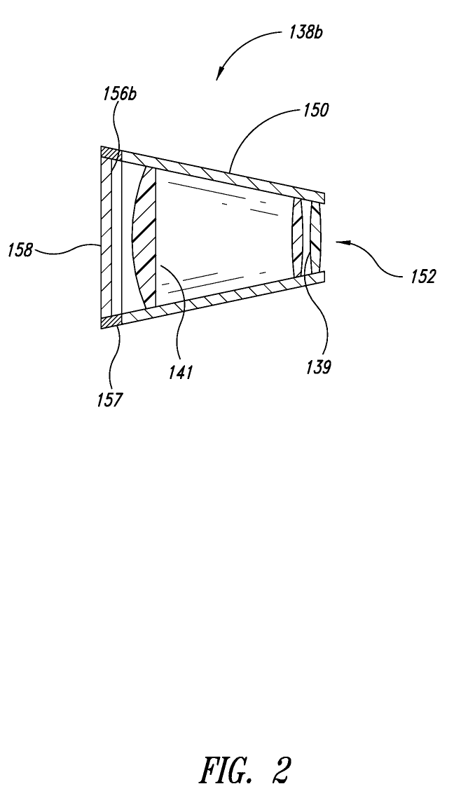

The imaging optics can be a passive system that functions without utilizing electrical power to provide an image. In some embodiments, including the illustrated embodiment of FIG. 2, imaging optics 152 of the loupe 138 transmit light rays to the left eye of the user. The imaging optics 152 includes eyepiece lenses 139 and an objective lens 141. A plurality of prisms or an erector can erect the image. For example, an erector can be positioned between the lenses 139, 141 to produce an upright image. Other types of imaging optics can also be used.

FIG. 2 also shows a housing 150 holding the imaging optics 152 and the filter 156b permanently or temporarily coupled to the housing 150. The filter 156b can be permanently coupled to the housing 150. Alternatively, the filter 156b can be detachably coupled to the housing 150 such that the filter 156b can be replaced with another filter having different characteristics selected based on the characteristics of the photoactivatable agent. A wide range of different types of filters can be used with the loupe 138b. Fasteners, pins, magnets, threads (e.g., internal/external threads), or other types of coupling elements can temporarily couple the filter 156b to the housing 150. In some embodiments, the filter 156b has an integral or separate outer body 157 that clips onto the housing 150. For example, the outer body 157 can be a generally tubular frame surrounding the filter 156 and can be magnetically coupleable to the housing 150.

The filter 156b can include, without limitation, one or more bandpass filters, narrow bandpass filters, longpass filters, shortpass filters, active filters, or passive filters, as well as other types of optical elements capable of selectively transmitting light having certain properties. The filter 156b may have a monolayer or multilayer construction and may pass wavelength(s) or waveband(s) associated with the fluorescence of the agent and attenuate other frequencies outside of those wavelength(s) or waveband(s). In some embodiments, the filter 156b is a passive bandpass filter that blocks all, or most of, the wavelengths outside of one or more selected wavebands. Passive filters are filters that function without utilizing electrical power and can include, but are not limited to, one or more dye filters, color filters, or other filters that continuously filter out unwanted light. These types of filters have excellent signal-to-noise ratios for clear images. In some embodiments, the filter is capable of transmitting at least about 80% of the fluorescence emission and at least about 80% of other light energy (e.g., light energy from the source 110) is inhibited (partially or completely blocked) from transmission. Longpass filters are useful for separating different colored light or wavelengths at least 100 nm or 150 nm apart. For example, a polyimide film can be used as a longpass filter. Polyimide has been shown to block the majority of excitation light from Talaporfin Sodium that is in a range of about 400 nm to about 440 nm and to allow transmission of fluorescence in a range of about 600 nm to about 700 nm. In some embodiments, for example, most or substantially all of a transmission of fluorescence in the range of about 600 nm to about 700 nm is transmitted through the filter. The filters discussed in connection with FIGS. 5-7 can be generally similar to the filter 156b, except as detailed below.

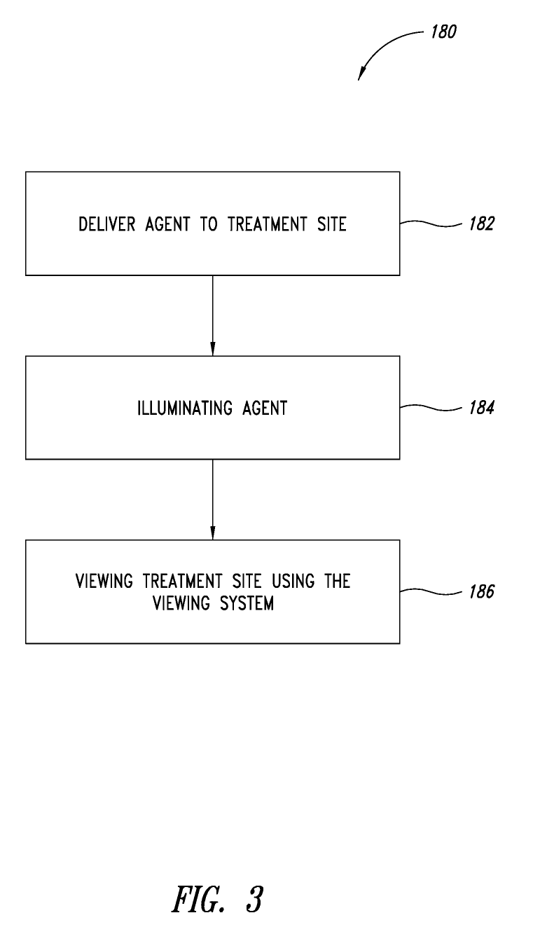

FIG. 3 shows a flow chart 180 of one method of using the system 100. Generally, the photoactivatable agent is delivered to the treatment site 120 such that, when the agent is illuminated by the light source 110, the agent fluoresces. The photoactivatable agent can accumulate in the targeted tissue 124 and can be partially or completely cleared from the tissue 122 prior to the illumination process. By looking through the viewing device 105 and exciting the accumulated photoactivatable agent, the observer can directly view and differentiate between the tissues 122, 124.

At 182, the photoactivatable agent is delivered to an individual. The agent can be administered locally (e.g., topically, via local injection, via a patch, or the like) and/or systemically (e.g., orally, intravenously, via an implant, or the like). Subsequently, the photoactivatable agent associates (e.g., accumulates, binds, absorbs, and/or links) to the tissue 124 more so than with the healthy tissue 122. U.S. application Ser. No. 09/905,777, which is incorporated by reference in its entirety, discloses various types of photoactivatable agents, delivery techniques, and associating techniques that can be used. Once the agent is bound to the tissue 124, the tissue 124 can be identified by administering light of an appropriate wavelength(s) or waveband(s) corresponding to the activation wavelength(s) or waveband(s) of the agent.

To systemically administer the photoactivatable agent, the agent may be formulated into suitable pharmaceutical preparations such as solutions, suspensions, tablets, dispersible tablets, pills, capsules, powders, sustained release formulations or elixirs, for oral administration or in sterile solutions or suspensions for administration, as well as transdermal patch preparation and dry powder inhalers. The photoactivatable agent can be administered in a dry formulation. The agent may also be administered in a liquid formulation, either alone with water, or with pharmaceutically acceptable excipients. The dose of agent will vary with the target cell(s) sought, the location and size of the treatment site 120, the subject's weight, and/or the timing of the surgical procedure. For example, an appropriate dose of the agent can be administered at least 12 hours, 24 hours, or 48 hours before performing a resection.

The photoactivatable agent can accumulate in the unwanted targeted tissue 124 such that the concentration of the agent in the tissue 124 is greater than the concentration of the agent, if any, in the tissue 122. For example, the agent can be configured to selectively bind with the tissue 124 and to not bind with tissue 122. Prior to viewing, the physician can allow a sufficient amount of time to pass so that the photoactivatable agent not bound to tissue 124 partially or completely clears from the treatment site 120, thereby leaving only agent bound to the tissue 124. U.S. Pat. No. 6,602,274, which is incorporated herein by reference in its entirety, discloses various methods of selectively binding photoactivatable agents to different types of tissues. The methods, agents, and delivery techniques disclosed in U.S. Pat. No. 6,602,274 can be used to selectively target different types of tissue.

The system 100 can also be used with different photoactivatable agents that are known in the art, and some of which are listed in U.S. Pat. No. 7,015,240, which is incorporated by reference with regard to disclosed photoactivatable agents and compositions. In some embodiments, the photo-activatable agent is comprised mostly or entirely of talaporfin sodium (mono-L-aspartyl chlorin e₆). Talaporfin sodium is a chemically synthesized photosensitizer, having an absorption spectrum that exhibits a maximum peak at about 415 nm and another absorption peak at 664 nm. Other types of photoactivatable agents that selectively fluoresce can also be utilized. In some embodiments, the agent has a peak energy absorption in the violet-blue sector of visible light ranging from about 400 nm to about 475 nm and outputs wavelengths in the red sector of visible light ranging from about 620 nm to about 750 nm. In some embodiments, the light incident from the light source can be blue light with wavelength(s) in a range of about 400 nm to about 430 nm (e.g., 410 nm) to excite the agent that emits a different wavelength or waveband, which allows for simple and inexpensive filtering. In some embodiments, one or more red light sources (e.g., sources that emit a wavelength of about 664 nm) can be used for deeper penetration for increased fluorescence as compared to other types of light, such as blue light.

In some embodiments, the photoactivatable agents include one or more targeting agents, including small molecule targeting agents, liposomal agents (e.g., liposomal spheres coated with a targeting substance), nanoparticles (e.g., carbon nanoparticles, iron oxide nanoparticles, or the like), antibodies, combinations thereof, or the like. In certain embodiments, the nanoparticles can be coated or otherwise associated with starch, chlorotoxin, or the like, or other targeting materials. Other types of targeting agents, including antibody targeting agents, can also be used. Many of these targeting agents are known in the art and are readily available.

At 184, the light source 110 illuminates the treatment site 120 to cause fluorescence of the tissue 124. The fluorescence can be in a different spectrum from the spectrum outputted by the light source 110 such that the fluorescence and reflected light are visually different. In some embodiments, the light source 110 emits blue or blue-violet light and the fluorescence of the agent is red, as noted above. The system 100 attenuates the blue or blue-violet light to increase the relative amount of red light viewed by the user, thus intensifying the red images corresponding to the tissue 124 to be removed. Because the system 100 attenuates certain wavelength(s) or waveband(s) and transmits other wavelengths (e.g., light that is not blue light or blue-violet light), tissue that does not fluoresce is also viewable. In some embodiments, the light energy from the light source 110 is different from the light energy emitted by the agent. This allows the system 100 to attenuate light energy from the fluorescing agent such that the user views both the fluorescing tissue and surrounding non-fluorescing tissue, even without an amplification system. For example, the color of light emitted by the agent can be different from the color of incident light from the light source 110. A wide range of different wavelengths can be used to generate a desired color of light emitted by different agents in order to allow convenient filtering. The user can observe any portion of the subject, including the surgical site and surrounding tissue, to navigate with respect to fluorescing targeted tissue and non-targeted tissue.

At 186, the user directly views the treatment site 120 by looking through the viewing device 105 and observes enhanced contrast between the tissue 124 containing the photoactivatable agent and tissue 122 that does not contain a significant concentration of the agent. The user can readily identify the tissue 124, including the margins of the tissue 124.

The surgeon can visually locate and inspect the tissue 124 to develop an appropriate surgical plan. While observing the treatment site 120, the user surgically removes the tissue 124. Because the system 100 provides three-dimensional real-time viewing, the user can more accurately manipulate a medical instrument (e.g., a surgical instrument) to navigate around healthy tissue 122 and non-targeted features that should remain intact. To reduce problems (e.g., eyestrain) often associated with pulsing or flickering light, the light source 110 can continuously illuminate the treatment site 120. For example, the light source 110 can be a LED to which electrical energy is delivered to illuminate the site 120 for a desired length of time.

The system 100 is also used to confirm whether the targeted tissue 124 has been removed from the subject. A wide range of different types of resection procedures can be performed to ensure complete tissue removal. Resection procedures can be the partial or complete surgical removal of diseased tissue from surrounding tissue, a diseased organ, or a diseased structure.

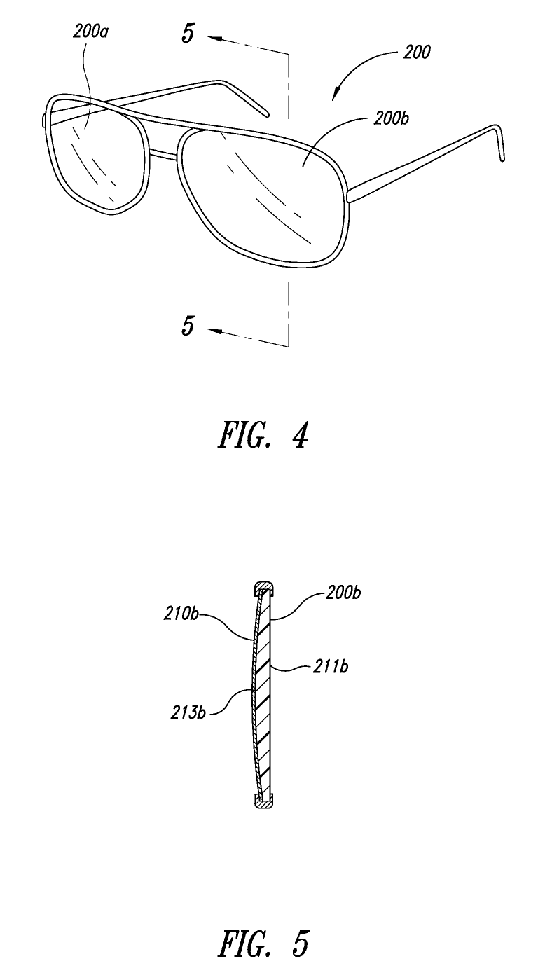

FIG. 4 shows a system 200 in the form of goggles. The term “goggles” is broadly construed to include, without limitation, a pair of eyeglasses worn to protect the eyes from hazards, such as blood spatter, flying tissue, liquids, glare, or the like. The goggles 200 have imaging optics 200a, 200b (collectively 200). Each of the imaging optics 200 may or may not provide vision correction and may be similar to one another, unless clearly indicated otherwise.

FIG. 5 shows the imaging optics 200b that includes a filter 210b that overlays a lens 211b. In some embodiments, the filter 210b is a sheet that is adhered to an outer surface 213b of the lens 211b. In other embodiments, the filter 210b is integrated into the lens 211b itself. For example, the lens 211b can be made of a material that attenuates certain wavelength(s) or waveband(s). The lens 211b can be a protective lens and/or a corrective lens. In some embodiments, the filter 210b is applied to conventional goggles used in surgery to form the system 200.

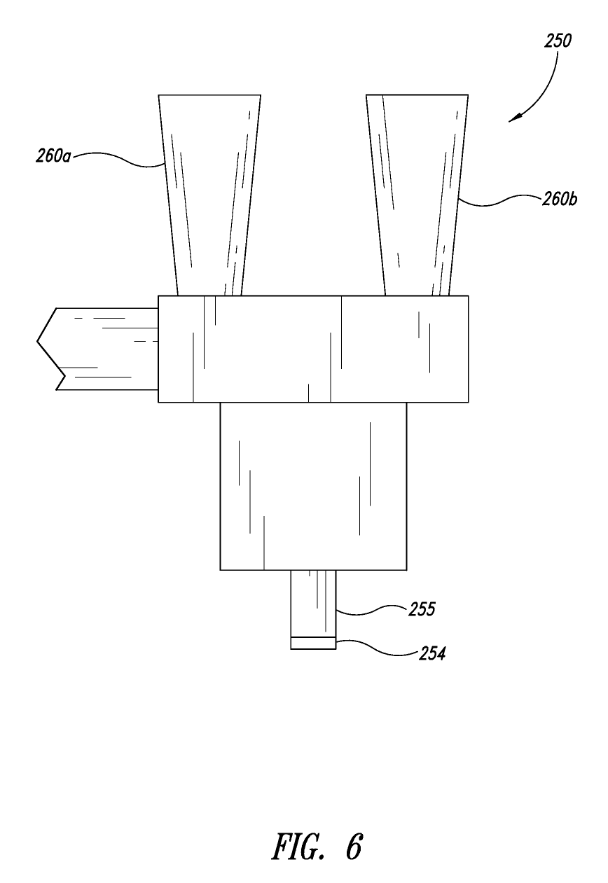

FIG. 6 shows a microscope 250 that includes an external filter 254 removably or permanently coupled to an objective 255. The microscope 250 can be a surgical microscope used during surgical procedures. The surgical microscope 250 can be part of a surgical apparatus that includes a stationary stand that allows controlled movement of the microscope 250 relative to a treatment site. A user can view a magnified image of a treatment site through the eyepieces 260a, 260b while the filter 254 enhances contrast between different types of tissue. Each eye of the observer therefore views an image that is produced, at least in part, by the filter 254. The configuration and optical properties of the filter 254 can be selected based on the design of the microscope. For example, the filter 254 can be integrated into one of the components of the microscope 250. In some embodiments, the filter is positioned inside of the objective 255. In some embodiments, each eyepiece has a filter. In some embodiments, the filter 254 threadably mates with a portion of a microscope. For example, the filter 254 can have internal threads that mate with complementary external threads of the microscope. Thus, the filter 254 can be screwed onto and off of the microscope.

FIG. 7 is a plan view of a surgical kit 300 that includes at least one container 302 holding a photoactivatable agent, a viewing device 304 (illustrated as a clip-on filter for eyewear or a microscope), and instructions for use 306. These items are contained within packaging 310, such as sterile packaging. The components of the kit 300 can remain sterile until the packaging 310 is opened to perform a procedure. The container 302 may be, without limitation, a bottle, pouch, packet, vial, or other type of object suitable for containing the photoactivatable agent. In some embodiments, the container 302 is a syringe that is pre-loaded with the photoactivatable agent. The viewing device 304 can be mounted on another component through which a user observes the treatment area. The design of the viewing device 304 can be selected based on the design of that component.

The illustrated kit 300 also includes a resection instrument 320. A resection instrument may include, but is not limited to, a surgical laser, a bipolar coagulator, a monopolar coagulator, an ultrasonic dissector, an ultrasonic aspirators, a cautery (e.g., a loop cautery), a scalpel, a cutting instrument, a curette, forceps, or any sort of hard or soft tissue removal instrument or device. In some embodiments, the resection instrument 320 may be a resection aid instrument, such as, without limitation, a suction device, a fluid irrigation device, a mirrored device, a tissue retractor, an endoscope, an optical fiber, a visualization system, or the like.

The light source and/or filter can be a separate device or can be incorporated into a surgical instrument, such as the resection instrument 320. The light source can be mated, connected, or integral to the surgical instrument 320. The light source, in some embodiments, is mated with a detector, magnification system, and/or other type of optical device. The illustrated resection instrument 320 includes an optical element 321, which includes a light source, filter, or both. Fluorescence can be detected visually or through a use of a detector, such as a spectrometer type detector, signal detector, and the like. Such a detector may have audible or visual feature capable of conveying signal strength information to a user. The system may further be incorporated into an instrument (e.g., an endoscope, a bronchoscope, and the like) and used during, for example, an open, semi-open, or closed operation.

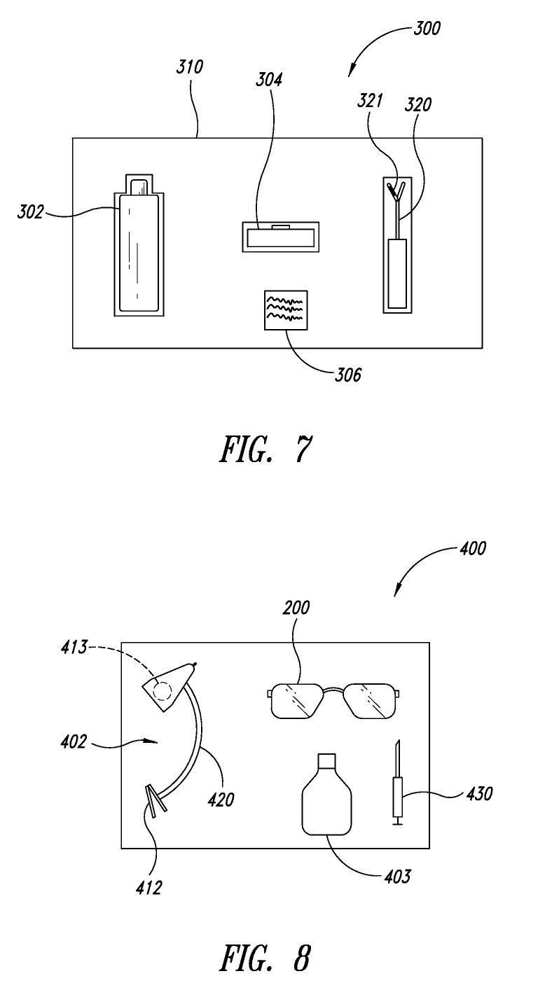

FIG. 8 shows a kit 400 that includes a lamp 402, a container 403 of photoactivatable agent, and a viewing device 200. The lamp 402 can be coupled to different types of equipment in an operating room using a clamp 412. A light source 413 (shown in dashed line as a light bulb) of the lamp 402 can be aimed so as to illuminate the treatment site. The illustrated lamp 402 is a gooseneck-type lamp with a flexible arm 420. The light bulb 413 may screw into a standard socket (or clip into a standard halogen socket). Additionally, the kit 400 can include one or more filters used with the lamp 402 to provide desired illumination. The illustrated kit 400 also includes a drug delivery device 430 in the form of a syringe.

Unless the context requires otherwise, throughout the specification and claims which follow, the word “comprise” and variations thereof, such as, “comprises” and “comprising” are to be construed in an open, inclusive sense, that is as “including, but not limited to.”

It should be noted that, as used in this specification and the appended claims, the singular forms “a,” “an,” and “the” include plural referents unless the content clearly dictates otherwise. Thus, for example, reference to a system that includes “a filter” includes a system with a single filter, or two or more filters. It should also be noted that the term “or” is generally employed in its sense including “and/or” unless the content clearly dictates otherwise.

Except as further described herein, the embodiments, features, systems, devices, materials, methods and techniques described herein may, in some embodiments, be used with or may incorporate any one or more of the embodiments, features, systems, devices, materials, methods and techniques described in U.S. Pat. Nos. 6,580,228; 6,602,274; U.S. application Ser. No. 09/905,777; and U.S. Provisional Patent Application No. 60/993,915. In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned U.S. Pat. Nos. 6,580,228; 6,602,274; U.S. application Ser. No. 09/905,777; and U.S. Provisional Patent Application No. 60/993,915.

The skilled artisan will recognize the interchangeability of various features from different embodiments disclosed herein. Similarly, the various features and steps discussed above, as well as other known equivalents for each such feature or step, can be mixed and matched by one of ordinary skill in this art to perform methods in accordance with principles described herein. Additionally, the methods which are described and illustrated herein are not limited to the exact sequence of acts described, nor are they necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, it is not intended that the invention be limited, except as by the appended claims.

1. A system for viewing targeted tissue at a treatment site, comprising:
a photoactivatable agent adapted to accumulate in targeted tissue;
a light source adapted to output an excitation wavelength that causes the photoactivatable agent to produce a fluorescence emission, the fluorescence emission having a fluorescence wavelength different from the excitation wavelength so as to allow separation of the excitation wavelength and the fluorescence wavelength; and
a direct-viewing device for observing the treatment site therethrough, the direct-viewing device including a filter adapted to separate the excitation wavelength and the fluorescence wavelength by filtering out the excitation wavelength from the light source that is reflected from the treatment site while allowing the fluorescence wavelength from the photoactivatable agent to pass therethrough to the eyes of a user to increase contrast between the targeted tissue and non-targeted tissue as the light source outputs light.

2. The system of claim 1, wherein the photoactivatable agent is capable of preferentially accumulating in the targeted tissue.

3. The system of claim 1, wherein the light source is capable of emitting at least one of coherent light or non-coherent light.

4. (canceled)

5. The system of claim 1, wherein the direct-viewing device has imaging optics configured to provide three-dimensional viewing of the treatment site.

6. The system according to claim 5, wherein the imaging optics are adapted to magnify an image of the treatment site viewed through the direct-viewing device.

7. The system of claim 1, wherein the direct-viewing device includes eyewear.

8. The system of claim 1, wherein the direct-viewing device includes at least one loupe.

9. The system of claim 1, wherein the direct-viewing device includes a surgical telescope.

10. The system of claim 1, wherein the direct-viewing device includes a surgical microscope.

11. The system of claim 1, wherein the filter comprises a bandpass filter or a longpass filter.

12. The system of claim 1, wherein the filter is a clip-on filter adapted to couple to a microscope and/or eyewear or the filter is adapted to threadably mate with a portion of a microscope.

13. The system of claim 1, wherein the light source is adapted to continuously output the excitation wavelength that causes the photoactivatable agent to produce a continuous fluorescence emission.

14. The system of claim 1, wherein the photoactivatable agent is capable of emitting red light when exited by blue light.

15. A resection kit for performing surgery, comprising:
instructions for use; and
packaging capable of holding the instructions for use and a system for viewing targeted tissue at a treatment site, the system comprising:
a photoactivatable agent adapted to accumulate in targeted tissue a photoactivatable agent adapted to accumulate in targeted tissue:
a light source adapted to output an excitation wavelength that causes the photoactivatable agent to produce a fluorescence emission, the fluorescence emission having a fluorescence wavelength different from the excitation wavelength so as to allow separation of the excitation wavelength and the fluorescence wavelength; and
a direct-viewing device for observing the treatment site therethrough, the direct-viewing device including a filter adapted to separate the excitation wavelength and the fluorescence wavelength by filtering out the excitation wavelength from the light source that is reflected from the treatment site while allowing the fluorescence wavelength from the photoactivatable agent to pass therethrough to both eyes of a user to increase contrast between the targeted tissue and non-targeted tissue as the light source outputs light

16. The resection kit of claim 15, further comprising:
at least one surgical tool configured to remove cancerous tissue identified by viewing through the direct-viewing device.

17. A system for viewing tissue at a surgical site, comprising:
a photoactivatable agent for activation by an excitation wavelength; and
an external viewing device through which a user is capable of observing the surgical site, the viewing device including a passive filter that transmits light emitted by the photoactivatable agent and that filters out the excitation wavelength to enable direct-viewing and fluorescence-enhanced optical differentiation of targeted tissue and other tissue at the surgical site.

18. The system of claim 17, wherein the viewing device further includes imaging optics configured to magnify an image of the surgical site and to allow rays of light from the surgical site to pass therethrough to the user's eyes.

19. The system according to claim 17, wherein the external viewing device is adapted to provide an analog image of the surgical site.

20. The system according to claim 17, wherein the external viewing device includes wearable goggles or a clip for coupling to a microscope.

21. The system according to claim 17, further comprising a light source carried by the viewing device.

22. The system according to claim 17, further comprising a resection tool carrying a light source adapted to cause fluorescence of the photoactivatable agent.

23. The system according to claim 17, further comprising a resection tool carrying a filter.

24. A surgical kit comprising:
instructions for use; and
packaging holding a system for viewing tissue at a surgical site, the system comprising:
a photoactivatable agent for activation by an excitation wavelength; and
an external viewing device through which a user is capable of observing the surgical site, the viewing device including a passive filter that transmits light emitted by the photoactivatable agent and that filters out the excitation wavelength to enable direct-viewing and fluorescence-enhanced optical differentiation of targeted tissue and other tissue at the surgical site.

25. A system for viewing tissue at a surgical site, comprising:
a photoactivatable agent capable of fluorescing; and
a filter that transmits light emitted by the photoactivatable agent while filtering out other light to increase a relative amount of the light outputted by the photoactivatable agent delivered to the user for true fluorescence-enhanced viewing of the surgical site enabling optical differentiation of targeted tissue for removal and other tissue at the surgical site.

26. The system of claim 25, further comprising a resection tool that carries the filter.

27. The system of claim 25, wherein the filter is adapted to threadably mate with a portion of a microscope.

28. The system of claim 25, wherein the filter is a clip-on filter adapted to couple to a microscope and/or eyewear.

29. A method of viewing targeted tissue at a treatment site, comprising;
delivering a photoactivatable agent to the treatment site of a subject;
exposing the photoactivatable agent at the treatment site to an excitation wavelength from an energizable light source to cause fluorescence of the photoactivatable agent at the treatment site;
directly viewing a fluorescence-enhanced image of the treatment site by looking through a viewing device, the viewing device including a filter adapted to filter out the excitation wavelength from the energizable light source reflected from the treatment site while allowing fluorescence to pass therethrough to the user; and
identifying targeted tissue at the treatment site based at least in part on directly viewing of the fluorescence-enhanced image.

30. The method of claim 29, wherein the energizable light source comprises at least one light-emitting diode positioned external to the subject.

31. The method according to claim 29, wherein exposing the photoactivatable agent at the treatment site to light comprises continuously emitting light.

32. The method according to claim 29, wherein directly viewing the fluorescence-enhanced image includes viewing the fluorescence-enhanced image with both eyes.

33. The method according to claim 29, wherein identifying targeted tissue includes locating margins of cancerous tissue by comparing visual differences between the cancerous tissue and non-cancerous tissue.

34. The method according to claim 29, further comprising removing cancerous tissue at the treatment site based, at least in part, on the identification of targeted tissue.

35. The method according to claim 29, further comprising accumulating the photoactivatable agent in cancerous tissue such that a concentration of the photoactivatable agent in the cancerous tissue is greater than a concentration of the photoactivatable agent in non-cancerous tissue.

36. The method according to claim 29, wherein the photoactivatable agent is configured to selectively accumulate in cancerous tissue.

37. The method according to claim 36, further comprising allowing sufficient time to pass for photoactivatable agent that is not accumulated to the cancerous tissue to clear from the treatment site prior to identifying the targeted tissue.

38. The method according to claim 29, wherein the fluorescence-enhanced image increases visualization of the fluorescence from the photoactivatable agent as compared to viewing by a naked eye.

39. The system according to claim 29, wherein the light source is adapted to output mostly blue light that causes the photoactivatable agent to output red light.

40. The system according to claim 29, wherein the photoactivatable agent comprises talaporfin sodium.

41. The system according to claim 1, wherein the filter comprises a first filter for the user's right eye and a second filter for the user's left eye.

42. The system according to claim 41, wherein the first filter is coupleable to a first loupe and the second filter is coupleable to a second loupe.

43. The system according to claim 1, wherein the filter is configured to filter out the excitation wavelength continuously emitted from the light source and reflected from the treatment site while allowing the fluorescence wavelength from the photoactivatable agent to produce continuous fluorescence-enhanced optical differentiation of the targeted tissue and other tissue at the surgical site.
